(19) United States
(12) Patent Application Publication
(10) Pub. No.: US 2016/0367332 A1
(43) Pub. Date: Dec. 22, 2016

(54) PROCEDURAL ASSIST DEVICE

(71) Applicants: Amit Navin Shah, North Potomac, MD (US); Curt Steven Kothera, Crofton, MD (US); Gregory John Hiemenz, Silver Spring, MD (US)

(72) Inventors: Amit Navin Shah, North Potomac, MD (US); Curt Steven Kothera, Crofton, MD (US); Gregory John Hiemenz, Silver Spring, MD (US)

(21) Appl. No.: 15/185,845

(22) Filed: Jun. 17, 2016

Related U.S. Application Data
(60) Provisional application No. 62/182,221, filed on Jun. 19, 2015.

(51) Int. Cl. A61B 90/11

(52) U.S. Cl. CPC A61B 90/11

(57) ABSTRACT

A device that assists surgical procedures by maintaining a specific percutaneous access location and orientation of needles, wires, trocars, dilators, catheters, etc., and which secures the tip of said access device(s) relative to said location and orientation during a percutaneous procedure. There are three main parts: (1) a base that conforms to the skin of the patient at various locations; (2) an adjustment mechanism mounted in ball-and-socket fashion inside the base and moving about at least one, and preferably multiple, axes of rotation relative to the base; and (3) a securement mechanism capable of being affixed to the adjustment mechanism for securely holding an access device at an angle to allow it to pass through the adjustment mechanism and enter the patient's skin at the desired angle and location. The device can be removed following the procedure without affecting the inserted access device(s).

### CROSS-REFERENCE TO RELATED APPLICATION(S)

The present application derives priority from U.S. Provisional Application Ser. No. 62/182,221 filed 19 Jun. 2015.

### BACKGROUND OF THE INVENTION

1. Field of the Invention

The present invention generally relates to assistive procedural devices and, more particularly, to procedures that are performed percutaneously with one or more procedural tools or devices that can benefit from maintaining a specific percutaneous access location, orientation of said procedural tools or devices, and securement of said procedural tools or devices.

2. Description of Prior Art

In placing a medical device or instrument into a patient it is best to use precision. However, correct device placement and preservation of device position are made difficult by various patient, disease, environmental, and user factors. Moreover, there is often a need for slight adjustment of the device in one or more dimensions (e.g., depth, angle with skin). Various systems, assemblies, devices, and methods for guiding and positioning, medical tools or devices are known in the medical field. The following are examples.

U.S. Pat. No. 4,265,561 to Heckele (herein, “Heckele '561”) discloses a device for holding a medical instrument wherein the device holder is mounted to a headband worn by the user. The device includes a base mounted to the headband with a recess therein, into which a rotatable member with a through-hole fits in a ball-and-socket fashion. The through-hole in the rotatable member accommodates an arm of the medial instrument.

U.S. Pat. No. 4,693,240 to Evans (herein, “Evans '240”) discloses a bone pin clamp as depicted in Evans ‘240.’ The bone pin clamp is secured in a ball-in-socket joint wherein the ball comprises a through-hole for receiving the pin (100). The through-hole may be axially grooved for securing the pin therein.

U.S. Pat. No. 6,328,748 to Henning (herein, “Henning '748”) also discloses a device for adjusting stereotactically and endoscopically located equipment where the holder comprises a lower ring (1) for receiving a ball (2) which will form a ball joint, and an upper ring (3) for locking the ball in a specific position, where the ball (2) has a channel for insertion of medical instruments, and where the lower ring (1) has an external threaded surface (8) for direct screwing to an area of a patient's skull. The device is characterized by the fact that the ball's (2) central point, and thereby the ball joint's fulcrum is arranged for positioning on a level with the patient's cranium by way of a groove-like or curved surface (21) on the holder which forms an abutment for the ball (2), wherein the groove-like surface (21) may be screwed directly into the patient's skull.

U.S. Pat. No. 7,204,840 to Skakoon et al. discloses (herein, “Skakoon '840”) discloses devices and methods to provide accurate targeting, placement, and/or stabilization of an electrode or other instrument(s) into the brain or other body organ. In an example an embodiment of Skakoon '840, a base 104 is secured to the skull using bone screws. A pedestal or tower 3002 is secured to base 104 as illustrated or, alternatively, is secured directly to the skull. Tower 3002 includes a socket 3004 housing a ball 3006. Ball 3006 includes a center opening that receives a rotating inner barrel sleeve 3008. In this example, sleeve 3008 includes one or more lumens 3010A-C extending there through for passing and guiding instruments, sheaths, stylets, etc. An affixation device, such as thumbscrew 3012, fixes the position of ball 3006 when the desired trajectory alignment has been obtained, such as by using the MRI, CT, PET, or frameless navigational guidance techniques discussed above. Proximal portions of lumens 3010A-C include recesses for snapping into place lips on devices inserted therein, such as alignment stem (or frameless adapter) 3014 and/or Luer stem 3016.

U.S. Pat. No. 7,824,417 to Magnusson et al. (herein, “Magnusson '417”) discloses a skin-mounted gimbal-like device for guiding a puncturing needle or other medical device to a treatment site. The device comprises a series of adhesive pads (6) that are releasably secured to the patient's skin and mounted with a base (5) and ball joint (7) to allow rotatable positioning of a needle guiding member (3) over the patient's skin.

U.S. Pat. No. 8,425,404 to Wilson et al. (herein, “Wilson '404”) discloses a laparoscopic surgical tool positioning device comprising a plurality of arms connected by ball-and-socket joints and able to be increased and decreased in length to provide the proper position and angle of skin entry for the laparoscopic tool. Wilson '404 discloses a clamp for holding the tool, comprising a spring-loaded clamping jaw (122) for gripping medical devices of different diameters. The clamping jaw is open on one side to allow releasable attachment to the device being positioned for skin entry,

U.S. Pat. No. 5,354,283 to Bark et al. (herein, “Bark '283”) describes a trocar retention device for attachment to the skin of the patient. The device comprises a trocar-receivable rotatable spheroid frictionally retained in a stabilizing member. The base, or stabilizing member (10) is preferably a frusto-conical member molded from a material such as silicone elastomer, rubber, plastic or the like and comprising a socket (14) into which the rotatable spheroid (22) sits and is frictionally engaged. The spheroid (22) has a cylindrical bore 24 that is adapted to slidably receive a trocar 22. In one disclosed embodiment, the stabilizing member (10) and the spheroid both have a slit (34) that allows the flexible material of the stabilizing member and the spheroid to be fitted over the trocar at the surface of the patient's skin after the has been placed in the proper position. The stabilizing member comprises a groove (28) around a “neck” portion thereof which may be secured by a clamp or other means to close the slit (34) and secure the stabilizing member and spheroid around the placed trocar. Bark '283 also describes bow the interior walls 26 of the stabilizing member 10 are inclined, preferably at a 45° angle to the skin of the patient, to prevent the trocar 22 from becoming dislodged during the surgical procedure by limiting the angle of inclination of the trocar 22. The spheroid is engaged and secured to the trocar (22) by the frictional force of the clamp or tie acting on the stabilizing member and spheroid. In some embodiments, the Bark '283 device is secured to the patient by means of a plurality of suction cups 40 positioned about the bottom of the base disc 18 of the trocar retaining apparatus. The suction cups 40 secure the stabilizing member 10 to the patient. The suction cups 40 provide for short term attachment of the stabilizing member 10 during surgery, rather than long term therapy. Alternatively, double sided. medical tape or a contact medical adhesive may be used to secure the stabilizing member 10 to the skin of the patient.

U.S. Pat. No. 4,841,967 to Chang et al. (herein, “Chang '967”) discloses a device for accurate positioning of needles for percutaneous insertion comprising a base having a circular angle indicator similar to a protractor for indicating the angle of needle insertion by reference to markings thereon. As seen in Chang '967, a needle holding device may comprise a needle holder 40 having a longitudinal grooved housing adapted to receive a needle 70 (or other elongated medical instrument intended for percutaneous insertion into a body cavity) in a longitudinal groove 45 defined in the front face 43 of housing 40. The needle 70 can be removably affixed to housing 40 by means of a removable clasp 50 which can be fabricated from a material and of a size such that the inner sides 65 of clasp 50 securely grip the sides 41 of housing 40 to hold needle 70 within groove 45 and to prevent needle 70 from moving.

### SUMMARY OF THE INVENTION

In accordance with the foregoing objects, the purpose of the present invention is to facilitate the performance of percutaneous procedures by stabilizing various access devices, thereby reducing trauma to the tissues (especially when the access devices have sharp tips) and enabling the user to take one hand off of the access device to perform other steps with two hands. Hence, the present invention is a device to maintain a specific percutaneous access location and orientation of access devices once said location and orientation have been identified by the user, and to secure the distal tip of said access device(s) relative to said location and orientation during said percutaneous procedure. Access devices include but are not limited to a needle, wire, trocar, dilator, and catheter (together referred to as “access devices”).

### BRIEF DESCRIPTION OF THE DRAWINGS

Other objects, features, and advantages of the present invention will become more apparent from the following detailed description of the preferred embodiments and certain modifications thereof when taken together with the accompanying drawings in which:

FIG. 1 is a top-front perspective view of a procedural assist device 1 according to a preferred embodiment of the present invention showing how a needle (e.g., access device) 100 interacts with it.

FIG. 2 is a back perspective view of the procedural assist device 1 of FIG. 1.

FIG. 3 is a bottom-front view of the procedural assist device 1 of FIGS. 1-2.

FIG. 4 shows a top perspective view of the preferred embodiment of base 10 and adjustment mechanism 40 of FIGS. 1-3.

FIG. 5 shows a back view of the base 10 and adjustment mechanism 40 of FIGS. 1-4, wherein said adjustment mechanism is rotated nearly parallel to said base.

FIG. 6 is a back view of base 10 and adjustment mechanism 40 of FIGS. 1-5, wherein said adjustment mechanism is rotated nearly vertical and then leftward with respect to said base.

FIG. 7 is a top view of the base 10 of FIGS. 1-6.

FIG. 8 is a back perspective view of the base 10 of FIGS. 1-7.

FIG. 9 is a back view of the base 10 of FIGS. 1-8.

FIG. 10 is a bottom perspective view of the base 10 of FIGS. 1-9.

FIG. 11 is a sequential operating illustration of the securement components 70 (shell 71 and detent plug 72).

FIG. 12A is a side cross-sectional view of the securement mechanism in a fully closed position.

FIG. 12B is a side cross-sectional view of the securement mechanism in a fully open position.

FIG. 13 is a front perspective view of the isolated plug 72.

FIG. 14 is a rear perspective view of the isolated plug 72.

FIG. 15 is a front perspective view of securement mechanism 70 with an optional door feature 82, wherein said door is closed.

FIG. 16 is a front perspective view of securement mechanism 70 with optional door feature 82 as in FIG. 15, wherein said door is open.

FIG. 17 is a bottom view of the procedural assist device 1 of the present invention housed around the hub of a needle 101.

FIG. 18 is a side view of the procedural assist device of the present invention housed around the hub of a needle as per FIG. 17.

### DETAILED DESCRIPTION OF THE PREFERRED EMBODIMENTS

For purpose of the present disclosure, the term “percutaneous” means via needle-puncture of the skin, and “percutaneous access device” means any medical device that employs a percutaneous access modality for a medical procedure. Percutaneous access and procedures frequently refer to catheter procedures such as cardiac catheterization, percutaneous transluminal angioplasty (PTA) ballooning, stent delivery, filter delivery, cardiac ablation, and peripheral vascular or neurovascular catheter procedures. Also included are procedures for percutaneous access of other body cavities such as parts of the gastrointestinal track, peritoneal cavity, pleural cavity, airway, abscesses, and other fluid- or air-containing cavities. Moreover, procedures that employ the Seldinger approach to access a space in the body generally fit into the category of percutaneous procedures. Other percutaneous procedures serve to access bone including intraosseous vascular access, vertebroplasty, and bone marrow biopsy. Still other percutaneous procedures are used to biopsy tissues. The procedures listed above are meant to serve as examples and are not an exhaustive list. The core functions of the device of the present invention are to provide the following:

(1) a securement mechanism for percutaneous access devices, wherein a component of the subject invention holds the access devices in place relative to the base and adjustment components, accommodates access devices of different sizes and diameters, maintains at least three positions (fully open, partially open, fully closed), enables the user's hand to come off of the access device, skin and/or procedural location, and does not materially limit amount of access device available for the procedure;

(2) an adjustment mechanism to maintain the location and orientation of the access device(s), wherein a component of the subject invention moves about at least one axis relative to the base, may lock to hold the position identified by the user, and may be rotated to align with the base so as to easily remove the device from the patient; and

(3) a base component designed to conform to the skin at varying locations (e.g., on flat, curved, indented or otherwise irregular surfaces), which may include adhesive, may serve as part of a sterile and/or antimicrobial dressing, may be easily replaceable, is easily removable from the skin (e.g., at the end of the procedure), has a small footprint, and accommodates the concurrent use of other procedural tools, including but not limited to scalpel, hook or retractor, ultrasound probe. Multiple techniques and/or approaches may be used to accomplish each of the functions. The detailed discussion and figures below describe limited embodiments of the device.

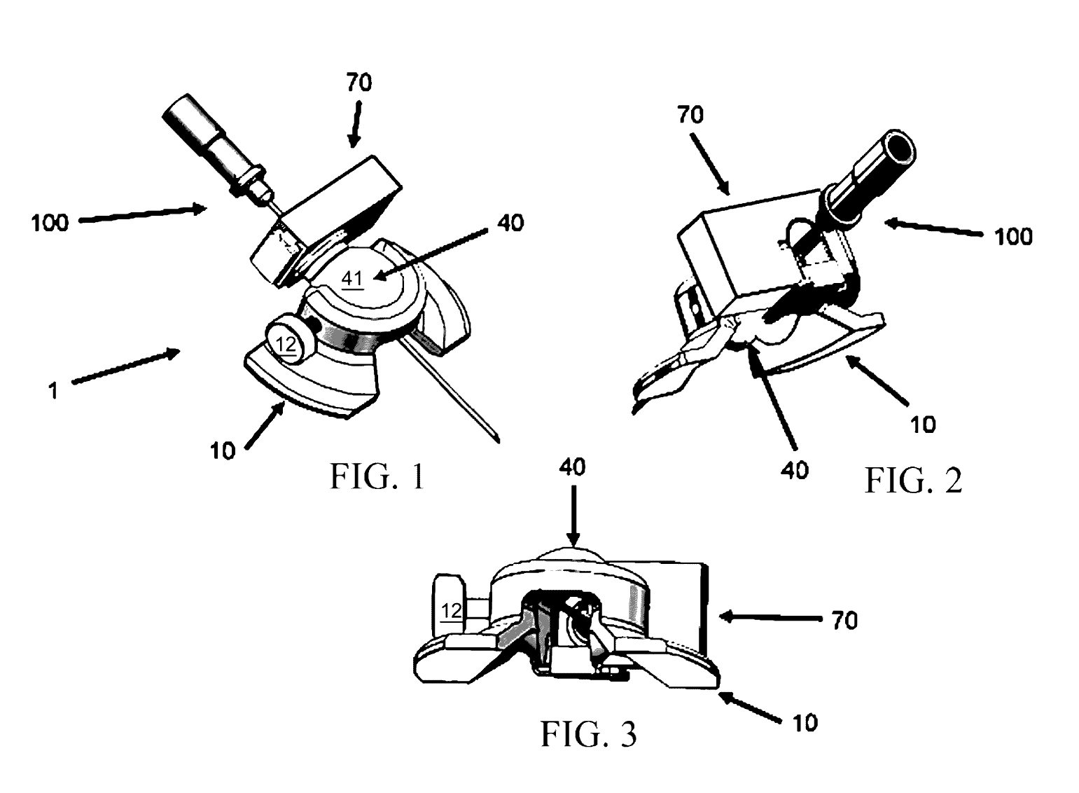

FIGS. 1-3 show three perspective views of a preferred embodiment 1 of the present invention and a needle 100. As shown and mentioned above, the device 1 is generally composed of a base 10, an adjustment mechanism 40, and a securement mechanism 70.

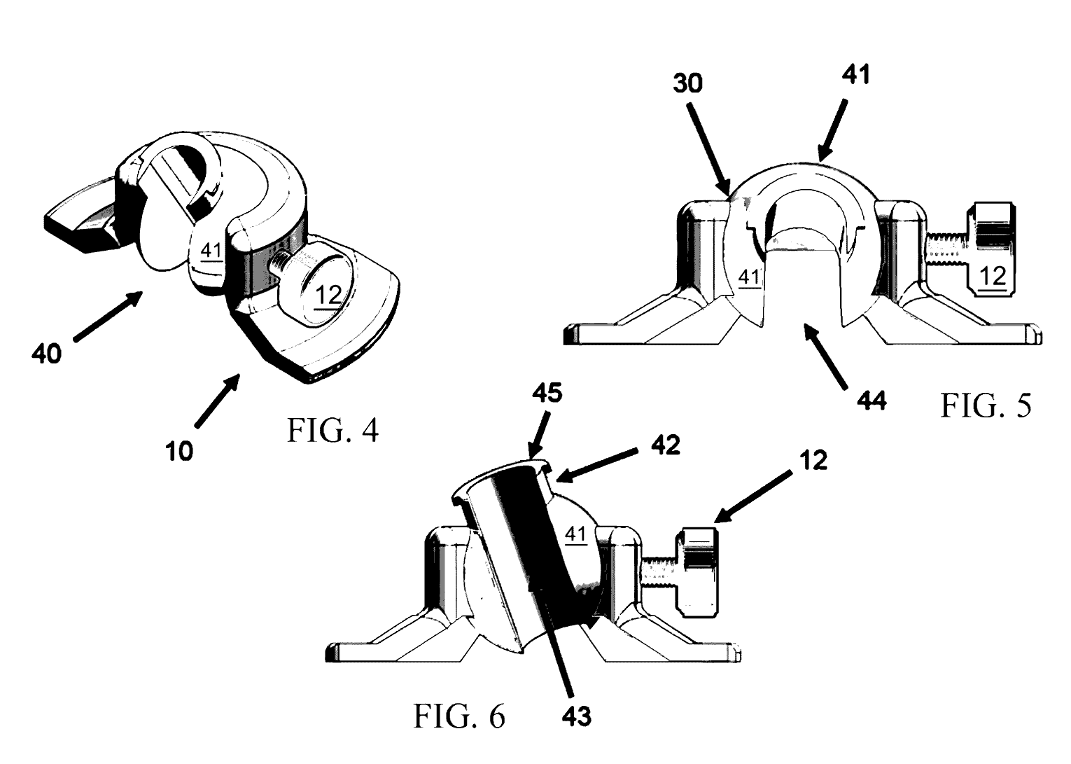

The device 1 features at least one axis of orientation adjustment relative to the identified procedure location on the patient, though the device preferably accommodates multiple axes of adjustment. This is herein accomplished by the adjustment mechanism 40 which articulates relative to base 10. FIGS. 4-6 show three perspective views of the preferred embodiment of base 10 and adjustment mechanism 40 of FIGS. 1-3, wherein adjustment mechanism 40 is shown in three different positions relative to base 10. The preferred adjustment mechanism 40 comprises a spherical, universal, or ball joint 30 providing free adjustment in all three rotational axes (roll, pitch, yaw). The joint 30 is preferably formed by providing adjustment mechanism 40 with a partial-spherical shaped body 41 seated within a conforming cavity 11 of base 10. The adjustment mechanism 40 can be angularly rotated within cavity 11 within a defined range of motion. It should be noted that other methods of adjustment other than a universal joint are possible without changing the scope or spirit of the present invention, including but not limited to at least one single axis pivot joint, any plurality of pivot joints, living hinge formed of gusseted semi-rigid material, or a flexible material. Fixation (setting or holding) of the desired angles is accomplished with a locking device 12. In the preferred embodiment, locking device 12 is a single, easily accessible thumb screw that is threaded through the base 10 and can be tightened against the spherical body 41 to maintain its orientation angles, though other methods of mechanical locking are obviously possible without changing the scope or spirit of the invention. Some of these include friction locks, clamps, clasps, etc. Note also that the locking device 12 (whether thumb screw or other locking mechanism) can preferably be attached to either side of the base 10 to better accommodate different handedness of the user. In the preferred embodiment with the locking device 12 being a thumb screw, this is accomplished by placing a threaded hole 19 on both sides of the spherical cavity 11 of base component 10.

The base 10 of the device 1 is configured to facilitate percutaneous access at acute angles of the access device(s) relative to the patient's skin (e.g., insertion angle approaching parallel to skin). The acute angles may range from nearly perpendicular as shown in FIGS. 4 and 6 to nearly parallel as shown in FIGS. 1-3 and 5.

A passageway 43 traverses the spherical shaped body 41 of adjustment mechanism 40, and exits through a collar 42 that protrudes from the spherical body 41 on one side. The percutaneous access device(s) are inserted through passageway 43 and collar 42, and so the orientation of the access device is intended to be substantially coaxial to the collar 42 and passageway 43 through adjustment mechanism 40.

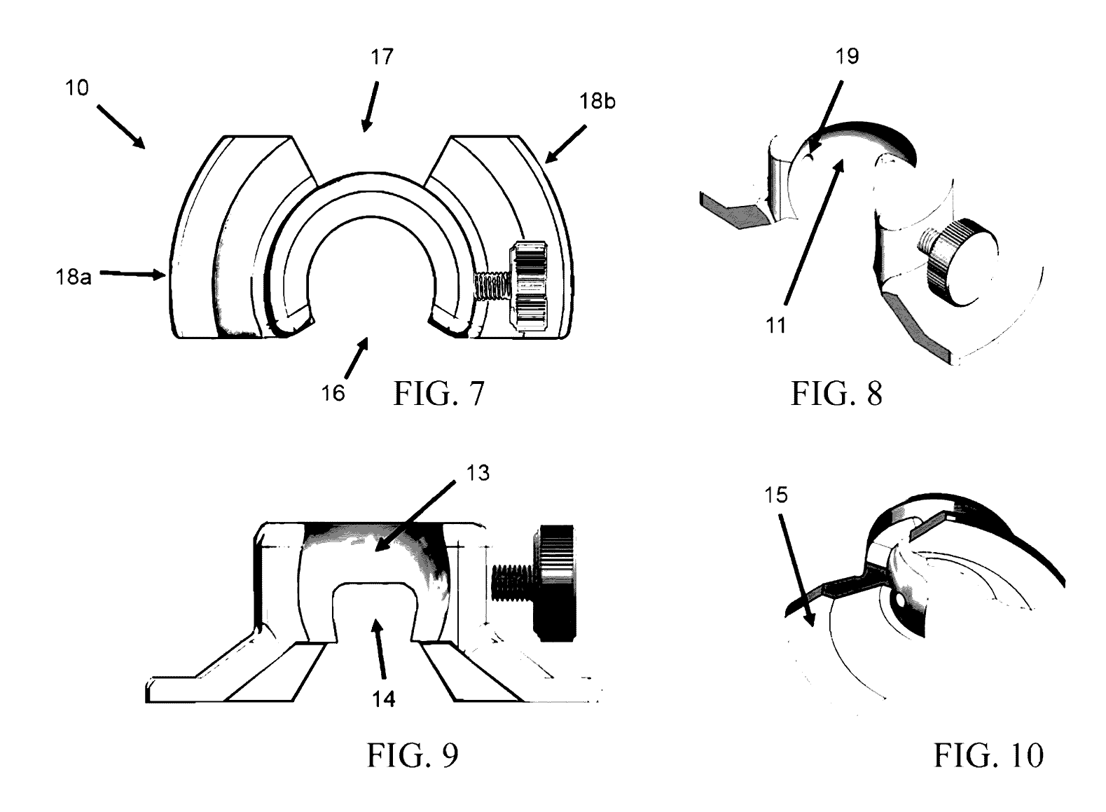

FIGS. 7-10 show four perspective views of the preferred embodiment of the base 10, which is preferably an annular member that includes a receptacle 13 for seating the spherical body 41 of adjustment mechanism 40, and feet 18 for elevating the receptacle 13. The percutaneous access device(s) are intended to pass freely through base 10 when they are in nearly all angles other than those corresponding to the feet 18, and if these angles are needed, base 10 can simply be re-positioned. As pictured in FIGS. 7-10 this is accomplished by making the receptacle 13 a discontinuous annulus, open on one side (at the back) and narrowing a portion of the receptacle 13 on the other side (at the front) so that it defines a notch 14. Notch 14 provides clearance for the access devices to pass. The under-side of the base 10 preferably defines a partial ring of enlarged diameter that is joined to the receptacle 13, the under-side of the ring providing angled feet 18 to increase stability and conformity to local skin topography at the procedure site. Note that feet 18 may be somewhat flexible to facilitate conformability. The notch 14 opens to a space 17 between the feet 18. The under-foot surfaces 15 are also preferably coated with an adhesive layer (e.g., peel-adhesive) to adhere the device to the patient's skin during the procedure for added stability, but the adhesive is preferably intended to create only a temporary bond and be easily removable, so as not to interfere with the inserted access device(s) when procedural assist device is removed. This bottom surface 15 may also provide and/or be part of a sterile and/or antimicrobial dressing at the procedure site and may be easily replaceable or exchangeable. Similarly, the base 10 and adjustment mechanism 40 are preferably configured to enable easy removal of the device 1 from the procedure site following completion of the procedure without interfering with the inserted access device(s). It can he seen in FIGS. 4-5 that the base 10 and adjustment mechanism 40 can be rotated to align their respective openings 16 and 44, so that the device 1 can be freely and unimpededly extricated from an access device passing therethrough. That is, neither the base 10 nor adjustment mechanism 40 form a complete circle or ring in at least one plane. Such an opening on the back side 17 of the base component 10 also allows use of other tools, such as a scalpel. These openings 16 and 17 in base 10 effectively form two feet 18a, 18b as seen in FIG. 7, the under-side of which contain surfaces 15. Note that securement of the access device(s) within the procedural assist device 1 is accomplished by the securement mechanism 70, as is described below.

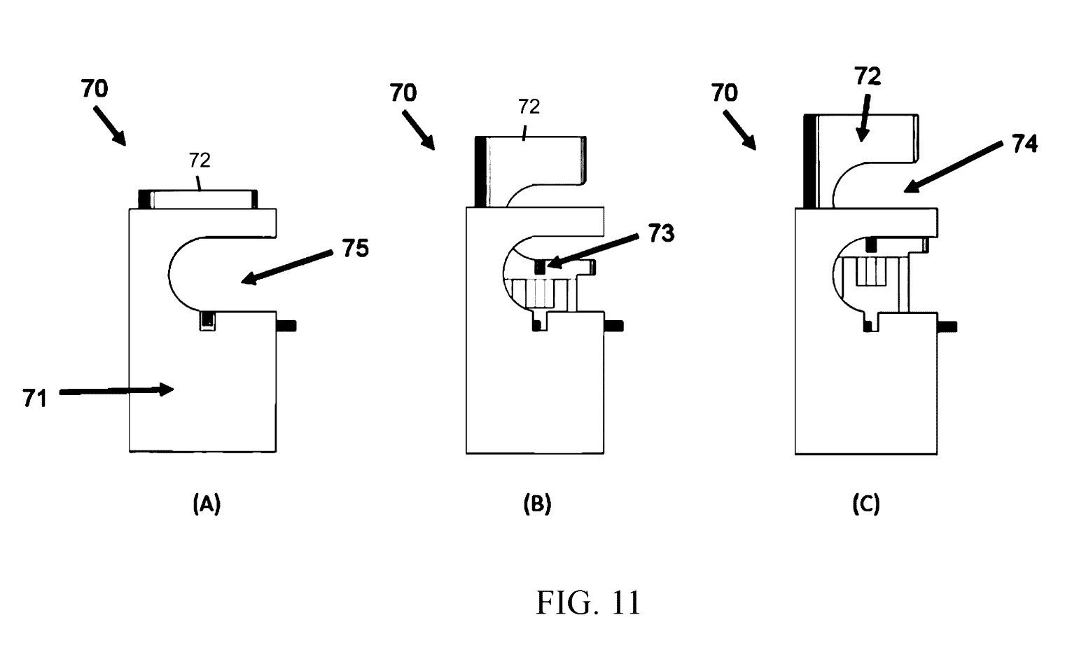

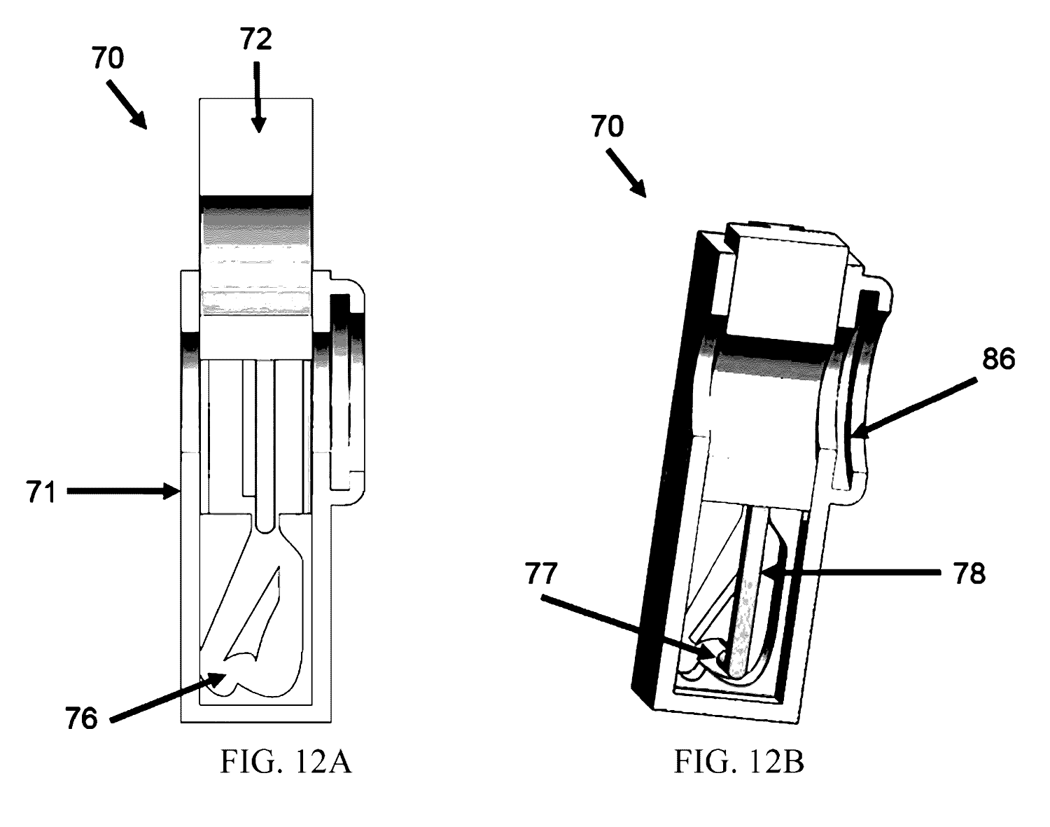

The adjustment mechanism 40 of the procedural assist device 1 is additionally fitted with securement mechanism 70 for holding the access device(s) in place relative to the base 10 and adjustment mechanism 40, as was shown in FIG. 1. FIG. 11(A-C collectively) shows the two primary securement components which are a shell 71 and detent plug 72, alone in three different positions: (A) fully open, (B) partially open, and (C) fully closed. These two components, shell 71 and detent plug 72, function essentially like a cord lock, where the detent plug 72 is pre-biased against the shell 71 to clamp access device(s) in jaws, but the plug 72 can be finger-pressed to open the jaws to remove the access devices. A spring (not pictured, but housed within shell 71) keeps an upward (relative to the figure orientation) force on detent plug 72 at all times such that the neutral position for this assembly is the fully closed position (C). The jaws are defined by conforming open C-shaped notches 74, 75 in both the detent plug 72 and shell 71. Small tabs 73 extend outward from the bottom of the C-shape 74 of the plug 72 to prevent the spring from pushing it completely out of the shell 71. It is this continual upward force that provides the securing function of the securement mechanism 70 about the access devices. If the top of the plug 72 (e.g., the button) is depressed any intermediate amount (anything less than all the way down), it will move back upward and hold the access device(s) when let go. Note that the spring stiffness is selected such that the reaction force does not crush, kink, or otherwise damage the access devices, and the partially open adjustability enables passage and securement of access devices of different diameters. That is, the size of the C-shaped jaws 74, 75 in the plug 72 and shell 71, respectively, can be chosen to allow passage of the largest access device. When the plug 72 is depressed completely, it will lock open. This is effectively a second neutral position. Then from this fully open position (A), when the plug is depressed that small amount farther and released, the plug 72 will return to the fully closed (C) or partially closed (B), yet securing an access device position. While three positions of these components are shown, there are essentially only two functional states: fully open, where the user need not maintain involvement with the procedural assist device 1 and access devices can freely pass through the aligned jaw openings (75, 74) and the passageway 43 through spherical body 41, and engaged (fully/partially closed), where the user need not maintain involvement with the procedural assist device 1 and access devices passing therethrough are securely held in place by interference from shell 71 and plug 72. It should be noted that other such methods of securement devices are included within the scope and spirit of the present invention, including but not limited to clothespin-like spring-loaded jaws and wire-winding/wrapping devices.

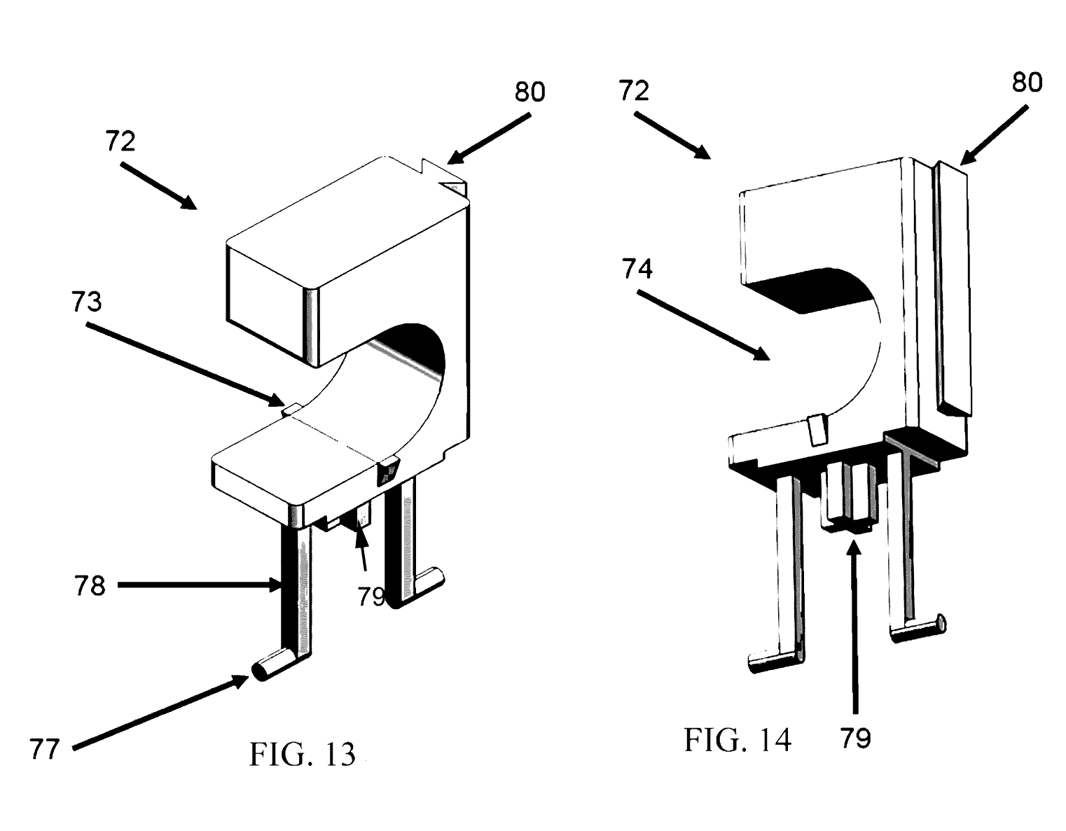

FIGS. 12A and 12B show two side cross-sectional views of the securement mechanism 70 to facilitate explanation of the fully open locking capability, in fully closed and fully open positions, respectively. As can be seen, opposing inner side faces of the shell 71 feature an inverted heart-like shaped track 76, through which pegs 77 on the plug 72 travel. These pegs 77 are more readily visible in FIGS. 13-14.

FIGS. 13 and 14 show front and rear perspective views of the isolated plug 72. When the plug 72 starts from the fully closed position and is pressed downward, the pegs 77 extending laterally from legs 78 follow the left side (FIGS. 12A and 12B) of the track 76 to the bottom, which moves the peg slightly to the right. When the plug 72 is released from here, the peg 77 moves upward slightly and to the right slightly along the track 76, locking the plug 72 in the fully open position. When the plug 72 is depressed from the fully open position, the peg 77 moves slightly down and slightly to the right again before following the right side of the track 76 back up to the starting position. Note that in the preferred embodiment, the pegs 77 are biased toward the right side of the plug 72, so when they start down the left side of the track, bending stiffness in their legs 78 will provide a continual rightward force to assist with the pegs 77 following the track 76 correctly until reaching their neutral (straight down) position, after which (toward the right), the track 76 is designed to guide the pegs 77 to their starting position. Note that the plug 72 component also has a spring centering feature 79 between the two peg legs 78.

Note that the back side of the plug 72 component also contains a slot-following feature 80, here being of a dovetail tongue-in-groove design, though this is not meant to be limiting. In a standard cord lock device, the shell component and plug component both make complete circles for other complete constraining shapes) such that the object passing therethrough can only pass through in that direction, e.g. O-shaped. The similar components of the present invention feature C-shaped jaws 74 and 75, as described, with the specific purpose of allowing the objects passing there through to exit the side unimpeded. This being the case, there is a tendency for the bottom of the C-shaped jaw 74 of plug 72 to protrude from the 75 of shell 71. To prevent this occurrence, the back side of the plug 72 has a guide feature 80 that interfaces with a companion guide feature 81 on the same side of the shell 71 to constrain the plug 72 motion to remain only up and down within the shell 71.

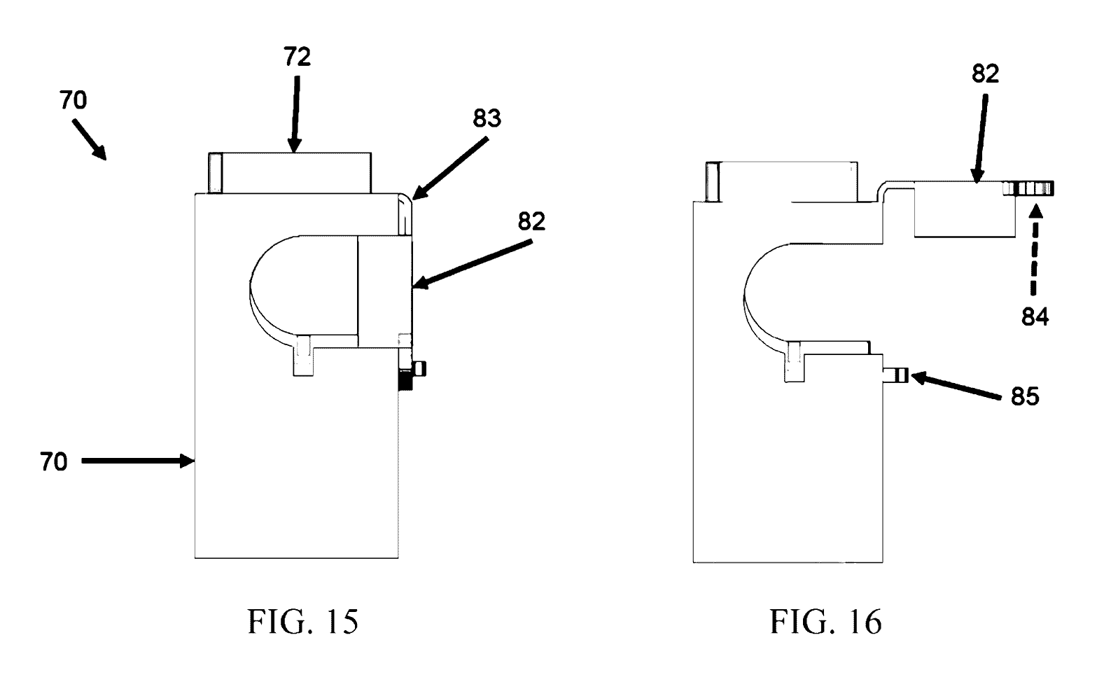

FIGS. 15 and 16 show front and rear perspective views of the isolated shell 71 with an optional door feature 82. To prevent the access devices from exiting the procedural assist device 1 through these C-shaped jaw openings 74 and 75, the shell 71 may be provided with a feature 82 that acts effectively as a door (e.g., transitioning between C-shape and D-shape) that remains closed until the user opens it at the time when ready to remove the procedural assist device 1 from the inserted access device. One embodiment of this door feature is visible in FIG. 1, but was removed from the subsequent figures for clarity. It is shown again more closely in FIGS. 15-16, where it can be seen that the door 82 preferably has a material or living hinge 83 on the top, connecting it to the shell 71, and a hole on the bottom, through which the locking tab 85 on the shell 71 holds it in place. Though a hinge design is shown here, an alternate embodiment may use a similar sliding dovetail design as on the back of the plug 80, where the door 82 is slid downward to open the side of the C-shaped jaw 75. Other hinged type connections are also possible within the scope and spirit of the invention.

In the preferred embodiment, the adjustment mechanism 40 can be rotated a certain way to fit in the opening 16 of base 10 for assembly. As pictured in FIG. 6, the upper collar 42 extending from the adjustment mechanism 40 is fitted with a lip 45 that interfaces with a matching slot 86 of shell 71 (see FIGS. 12A-12B) to facilitate assembly. Other attachment or assembly methods may likewise be employed without changing the invention, including but not limited to bonding, threading, and clamping.

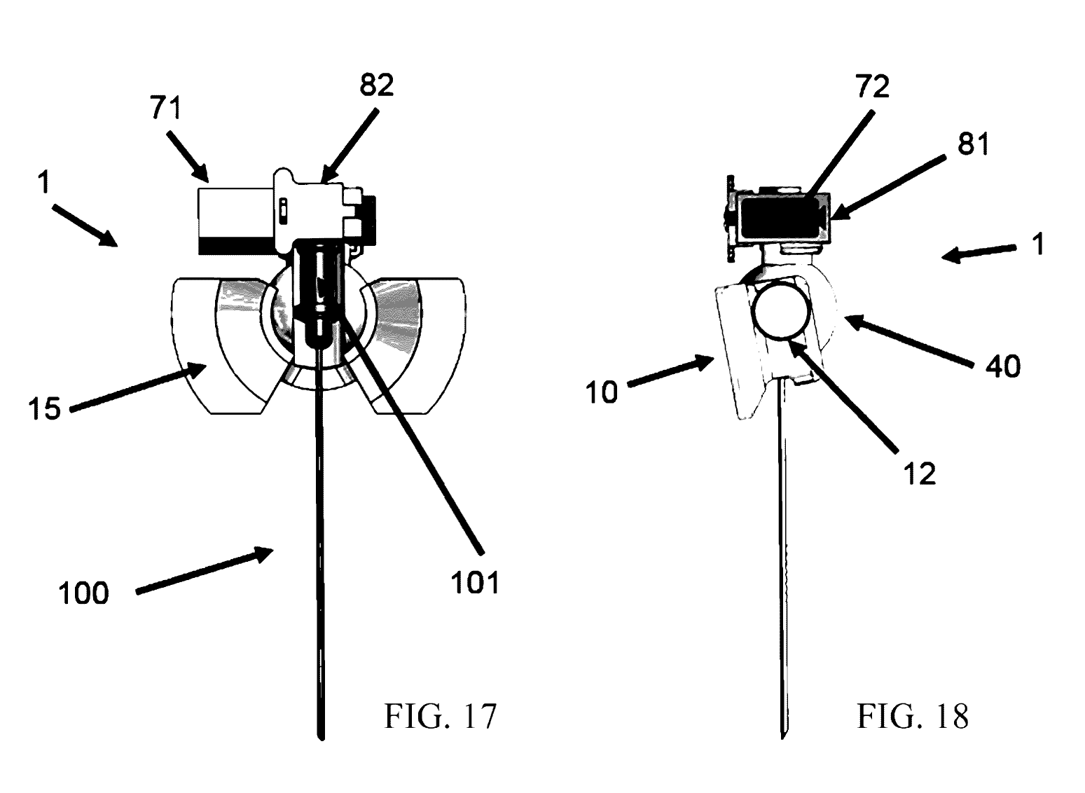

FIGS. 17 and 18 are a bottom and side view, respectively, of the procedural assist device 1 of the present invention housed around the huh of a needle 101. The procedural assist device 1 of the present invention is of minimal height, where the assembled device components have an axial length that is similar to the hub 101 of a needle 100 (FIG. 17). Note that a needle 100 is the first access device used in many percutaneous procedures, such as those that employ the Seldinger technique. As such, the passage way diameter of the procedural assist device 1 components is large enough to allow the needle hub 101 to pass there through. Further, the percutaneous procedure may be maximally facilitated with the present device by beginning the procedure with the device 1 positioned around the needle hub 101, where it can then be passed downward onto the patient at the correct location and orientation and secured in place with the features described above. The overall footprint of the device 1 is also small to enable concurrent use of other procedural tools, including but not limited to scalpels and ultrasound probes.

Having now fully set forth the preferred embodiment and certain modifications of the concept underlying the present invention, various other embodiments as well as certain variations and modifications of the embodiments herein shown and described will obviously occur to those skilled in the art upon becoming familiar with said underlying concept. It is to be understood, therefore, that the invention may be practiced otherwise than as specifically set forth in the appended claims.

What is claimed is:

1. A device for assisting with placement of a percutaneous device during a percutaneous procedure, comprising:
a base having an under-surface configured to conform to the skin, and a body defining a receptacle;
an adjustment mechanism configured to engage said percutaneous device, said adjustment mechanism comprising a body seated in the receptacle of said base and articulated therein for adjusting location and orientation of said percutaneous device relative to said skin; and
a securement mechanism attached to said adjustment mechanism for releasably locking said percutaneous device within said adjustment mechanism.

2. The device of claim 1, wherein the body of said base comprises an open slot for allowing removal of said device for assisting with placement from a patient without interfering with said percutaneous access device.

3. The device of claim 1, wherein said adjustment mechanism comprises a body formed in a partial sphere with an opening, said opening of said spherical body allowing removal of said device for assisting with placement from a patient without interfering with said percutaneous access device.

4. The device of claim 1, wherein said securement mechanism comprises a clamp with at least one opening, for allowing removal of said device for assisting with placement from a patient without interfering with said percutaneous access device.

5. The device of claim 2, wherein the body of said base has a notched-opening substantially opposite to said open slot opening.

6. The device of claim 5, wherein said base has opposed feet for temporary attachment to the skin of the patient, said base under-surface being beneath said feet.

7. The device of claim 6, wherein said under-surface is any one of a flat, curved, pliable or irregular surface.

8. The device of claim 1, wherein said base includes adhesive means on said under-surface or temporary attachment to the skin of the patient.

9. The device of claim 1, wherein said base includes a sterile and/or anti-microbial dressing on said under-surface.

10. The device of claim 1, wherein said base is easily removable from the skin of the patient.

11. The device of claim 1, wherein said adjustment mechanism has a length substantially equal to a length of a hub of a conventional needle.

12. The device of claim 1, wherein said adjustment mechanism allows movement about at least one axis relative to said base.

13. The device of claim 1, wherein said base and adjustment mechanism comprise at least a partial ball-and-socket joint.

14. The device of claim 1, further comprising a locking component for selectively locking said adjustment mechanism in a set position relative to said base.

15. The device of claim 4, wherein said openable clamp of said securement mechanism is adjustable to accommodate differently-sized percutaneous access devices.

16. The device of claim 4, wherein said securement mechanism has a plurality of operational positions.

17. The device of claim 16, wherein said plurality of operational positions include fully open, partially open, and fully closed.

18. The device of claim 1, wherein said securement mechanism is configured to secure a percutaneous access device by spring loading.

19. The device of claim 4, wherein said opening of said securement mechanism has a closable feature.

20. The device of claim 1, wherein said securement mechanism is comprised of a plug inserted in a shell.

21. The device of claim 20, wherein said plug is slidably constrained within said shell.
